(19) United States
(12) Patent Application Publication
(10) Pub. No.: US 2015/0038965 A1
(43) Pub. Date: Feb. 5, 2015

(54) METHOD AND APPARATUS FOR FRACTIONAL NON-INVASIVE SKIN TIGHTENING

(71) Applicant: LUMENIS LTD., Yokneam ILIT (IL)

(72) Inventor: Yoni Iger, Yoqneam (IL)

(21) Appl. No.: 14/445,534

(22) Filed: Jul. 29, 2014

Related U.S. Application Data
(60) Provisional application No. 61/860,091, filed on Jul. 30, 2013.

(51) Int. Cl. A61B 18/14

(52) U.S. Cl. CPC A61B 18/14; A61B 2018/00589
USPC 606/49

(57) ABSTRACT

Apparatus is disclosed for providing a RF fractional treatment to the skin tissue by providing parallel elongated electrodes on which conductive areas and non-conductive areas are provided. The providing of conductive and non-conductive areas assists in sparing enough essential healthy tissue components like blood capillaries to support intense wound healing responses.

### RELATED APPLICATIONS

This application is related to and claims priority to U.S. Provisional Application No. 61/860091, filed 30 Jul. 2013, which is herein incorporated by reference in its entirety.

### TECHNICAL FIELD

The present invention relates to a device and method for fractional tissue treatment and more particularly, to a non-invasive fractional skin tightening device and method.

### DISCUSSION OF THE RELATED BACKGROUND

Just a few years ago, the choice for treatment of skin laxity, which is a major aspect of the aging process, was only surgery. Today, non-invasive and minimally invasive skin tightening techniques are commonly used procedures for the treatment of loose skin of different anatomies. In order to produce a tightening effect non-surgically, methods for deep heating the dermis and possibly the fibrous septae supporting the dermis and subcutaneous fat to the underlying fascia have been developed. Heat, above a certain threshold, is known to coagulate and destroy collagen contained in target tissue, a phenomena characterized by collagen shrinkage. Over time, a wound healing response is initiated followed by collagen remodeling characterized by an overall result of a visible tightening effect.

The prior art discloses the use of infrared light which may be produced by a laser which can then target specific absorption bands of well-localized chromophores in the skin. Optical energy is then transformed into heat mainly by water contained in the tissue acting as a photon absorption agent. As a result, the energy is dispersed to a three dimensional volume of tissue at a controlled depth. Wavelength selection, varying parameters of the laser energy together with epidermal cooling, may control the effective depth of the treatment within the skin. Alternatively, infrared light may be generated by an intense pulsed light device to target water as chromophore in order to achieve uniform heating over three dimensional volume of the skin. However, due to high water concentration in target tissue and strong optical absorption by the water, optical-based skin tightening devices are limited to penetration depths of about 1-2 mm. Moreover, the entire bulk of the target tissue is being evenly treated. No un-treated tissue fragments within a target tissue are left. As a result, a target tissue may lack critical agents responsible for producing an effective and enhanced healing response.

Also known in the prior art is the monopolar radiofrequency technology (MRF) for skin tightening. MRF heats the tissue based on the natural tissue's resistance to the movement of ions within the RF field (Ohm's law). Unlike light energy, RF technology is based on an electric current that generates heat through the impedance of the dermis and subcutaneous tissue. A capacitive coupling membrane between the RF electrode and the skin transforms RF to a volumetric deep tissue-heating technology, rather than a concentrated point of heating source. This allows large amounts of energy to be distributed over a three-dimensional deeper volume of dermal tissue causing collagen denaturation followed by neo-collagenesis.

Also known in the prior art is an RF procedure which is applied to the skin by two or more bipolar electrodes. A conductive coupling medium may be applied between the skin and the electrodes to improve electrical coupling. The treatment electrodes are positioned on the skin surface facing a target dermal tissue area. The skin surface may be mechanically manipulated by an applicator so that a target dermal tissue may be inserted or folded between the electrodes. Typically, this is accomplished using an inverted cup-like device into which the tissue may be sucked using, for example, the application of a vacuum within the cup-like device. Such an applicator may position the RF electrodes in different orientations versus a target tissue. The RF current travels between the electrodes via the skin tissue, while impinging the tissue content. The tissue's electrical impedance transforms this current into heat. Above certain temperature and time thresholds, the heat causes coagulation of the tissue at a target volume. This in turn results in contraction of the tissue, revealing a tighter, less loose, skin phenotype.

RF emitting electrodes may be designed to have a typical length and distance between each other which together defines a target dermal zone to be treated. As a result, a three dimensional volume coagulation zone is formed within a target dermal area resulting in skin contracture. Tissue coagulation is more dominantly used in the elderly population to achieve tightening tissue effects rather than collagen synthesis which is more dominant for use in younger population. Like previously discussed technologies, this coagulated dermal zone is even and spares no healthy tissue in target tissue which is useful to intensively support a healing response following treatment. These same drawbacks are also applied with the more recently developed multi-source phase-controlled RF technology.

Fractional irradiation technologies are also known in the art. However fractional light-based technologies are limited to almost the same depths as discussed above for light treatment. Although higher energy may be delivered per treatment spot in a fractional pattern, in comparison to a uniform illumination, tissue water content and absorption characteristics still strongly dominate and limit depth of penetration.

Fractional bipolar RF technologies are also known in the prior art. This technology provides an array of micro-electrodes, each of which has a diameter of about 200-300 microns, to create superficial micro-ablative and coagulation effects adjacent to the contact area of each micro-electrode with the skin surface. Fractional RF has been used mainly for skin resurfacing-type of rejuvenation since less than 1 mm in depth thermal injury is formed overall under and in immediate proximity to each micro-electrode, therefore causing no skin tightening effect. The area of skin in direct contact with the micro-electrodes or adjacently below the micro-electrodes is selectively treated whereas the areas between the target areas are left intact.

It is the subject of this invention to overcome the above mentioned limitations and provide an effective skin tightening technology which can target and treat deep tissue volumes while sparing enough essential healthy tissue components to support intensive wound healing response.

### SUMMARY OF THE PRESENT INVENTION

The present invention discloses in one embodiment an apparatus for treating skin tissue with fractional RF energy which includes two elongated electrodes. The elongated electrodes are separated from one another by a distance when placed on the skin tissue; each of the electrodes may have one or more conducting areas and one or more non-conducting areas therealong. In addition, the conducting areas of one elongated electrode may be aligned with the conducting areas of a second elongated electrode; a source of RF energy for providing RF energy to the conducting areas of the elongated electrodes may be included. Upon application of RF energy to the one or more conductive areas, multiple separated RF fields within the tissue are created to provide fractional coagulation-based skin tightening.

In another aspect the apparatus may further include a cooling medium which may be applied to one or more of the electrodes for cooling the skin tissue.

In yet another aspect, the one or more conducting areas and the one or more non-conducting areas are placed alternatively on the one or more elongated electrodes; the elongated electrodes may be placed on the skin tissue substantially parallel to one another.

In another aspect, a method is disclosed for treating skin tissue with fractional RF energy in which two elongated electrodes are provided and are separated from one another by a distance when placed on the skin tissue. Each of the electrodes may have one or more conducting areas and one or more non-conducting areas placed therealong. The conducting areas of one elongated electrode may be aligned with the conducting areas of a second elongated electrode. RF energy may be provided to one or more of the conducting areas of the elongated electrodes. Upon application of RF energy to the one or more conductive areas, multiple separated RF fields within the tissue may be created to provide fractional coagulation-based skin tightening.

### BRIEF DESCRIPTION OF THE DRAWINGS

FIGS. 1A and 1B illustrate prior art RF skin tightening devices.

FIG. 2 illustrates one embodiment of the present invention.

### DETAILED DESCRIPTION

Prior to setting forth the detailed description, it may be helpful to set forth definitions of certain terms that will be used hereinafter.

The term “electrode” as used herein in this application refers to any type of energy transmitting element or energy irradiating element or energy delivering element. These elements may include, among other things, radiofrequency electrode, light emitting diodes, laser diode, optical fiber or ultrasound transducer. An array of electrodes or conductive areas or multiple electrodes may include one or more types of electrodes as defined above.

The term “fractional treatment” as used herein in this application refers to a treatment of target tissue or organ in which at least one treatment point is created in the tissue and is surrounded by a non-treated tissue. On a target tissue, one or more treatment points may be created in a variety of sizes, depths, patterns and densities. Fractional treatment may be invasive, non-invasive or a combination of the two.

The term “energy source” as used herein in this application refers to any energy source which may create fractional treatment. Non-limiting examples for such energy sources are lasers, non-coherent light sources, radio frequency generators, microwave generators, cryogenically cooled material, ultrasound etc.

With specific reference now to the drawings, it is stressed that the particulars shown are by way of example and for purposes of illustrative discussion of the preferred embodiments of the present invention only, and are presented herein to provide what is understood to be the most useful and readily understood description of the principles and conceptual aspects of the invention. In this regard, no attempt is made to show structural details of the invention in more detail than is necessary for a fundamental understanding of the invention. The description taken with the drawings make apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated in the drawings. The invention is applicable to other embodiments or of being practiced or carried out in various ways. Also, it is to be understood that the phraseology and terminology employed herein is for the purpose of description and should not be regarded as limiting.

Essential organs such as healthy tissue or blood capillaries located between necrotic zones which are created deep in the skin, in depths bigger than about 1-2 mm, increase blood perfusion to more superficial papillary dermis and epidermis around them and are essential for maintaining an effective and intensive wound healing response post coagulated-based skin tightening. Improved tissue perfusion may overcome the lack of cellular reservoir within coagulated zones which may result in an accelerated skin aging in situ.

It is therefore one aspect of the present invention to provide a non-invasive fractional skin tightening device and method of treatment.

It is another aspect of the present invention to create non-invasively a fractional pattern of multiple and discrete deep coagulated zones, at depths higher than about 1-2 mm, by applying an array of multiple bipolar RF electrodes and while keeping the skin surface intact. Such a device will interrupt the continuity or uniformity of the bulk coagulated-tightened zone taught by the prior art.

It is therefore another aspect of the present invention to keep portions of vascular bed or tissue within coagulated target tissue intact as well as to maintain intact cellular reservoir islets between coagulated deep treatment zones to initiate and support an intensive and effective healing response.

It is another aspect of the present invention to provide a fractional bipolar RF skin tightening device and method of treatment involving a capacitive coupling material to reduce current cross-flow between electrodes.

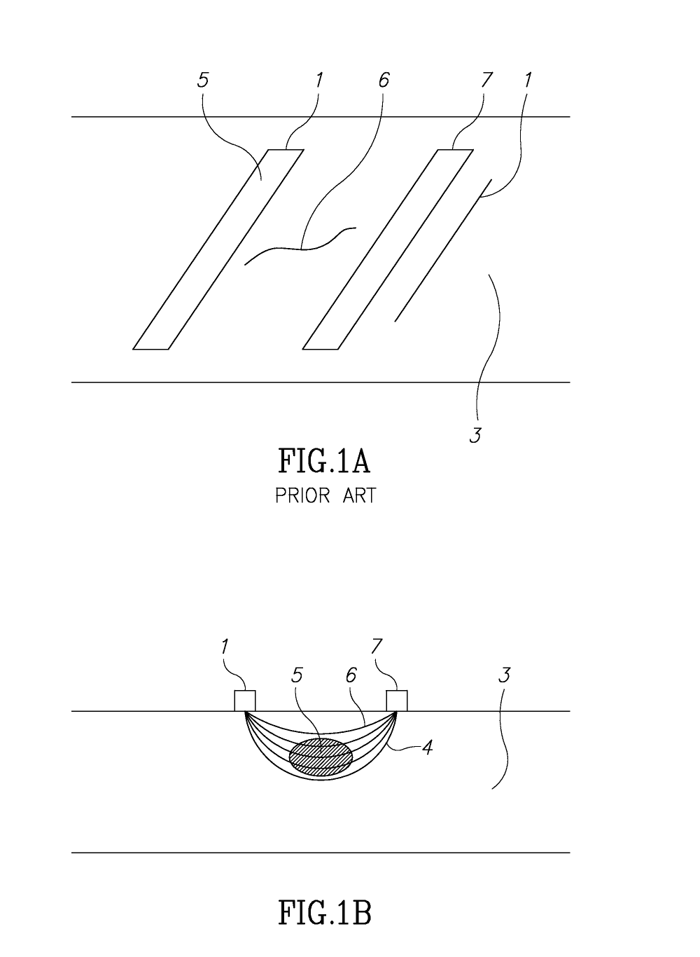

FIGS. 1A and 1B illustrate an RF skin tightening device as described in the prior art. An applicator (not shown) is configured to position bipolar electrodes 1 and 2 on skin tissue 3. The applicator is configured to be connected to an RF generator. During treatment, electrodes 1 and 2 are coupled to skin 3 and deliver a RF energy field 4 into a target tissue. A cigar-shaped coagulated zone 5 is created deep in the tissue having a longitudinal axis which is parallel to the longitudinal axis 7 of the electrodes. Blood capillary 6 which is located within the target tissue to be treated will be coagulated and damaged during treatment, as can best be seen in FIG. 1B. As a result, perfusion of tissue external to the treated tissue and supported by this blood capillary will be decreased and therefore wound healing response will be suppressed. The aging process may also be accelerated.

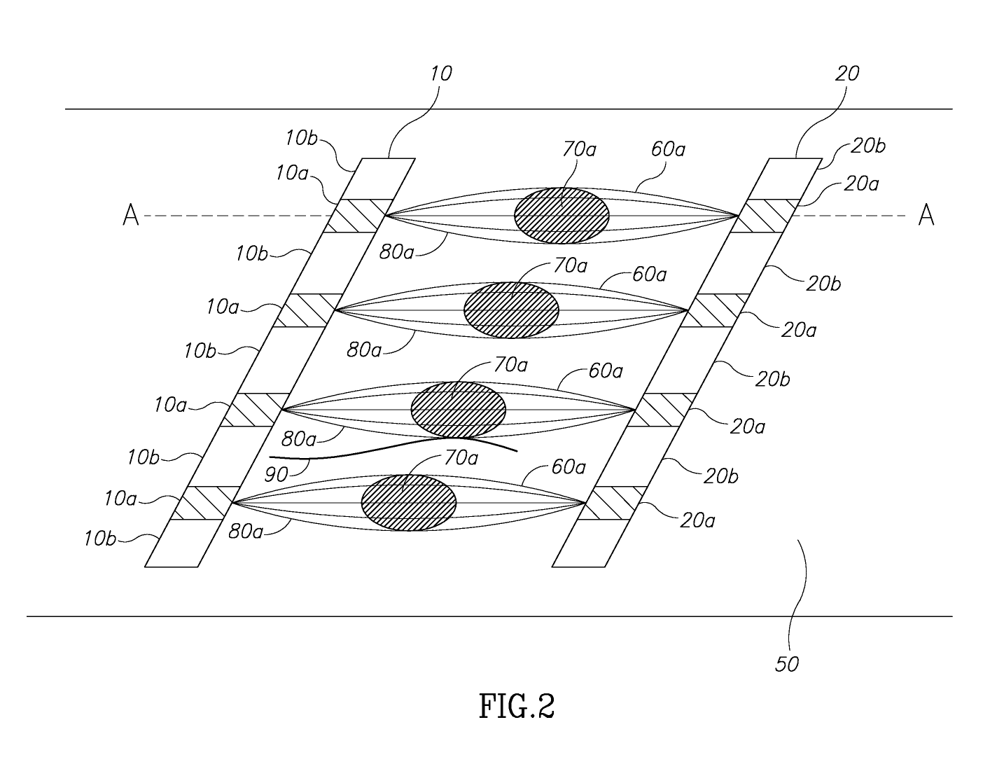

FIG. 2 illustrates one configuration of an electrode array according to one embodiment of the present invention. An applicator (not shown) is configured to position at least two bipolar electrodes 10 and 20 on skin 50. Each electrode may have at least two conductive areas separated by at least one isolating area. Electrode 10 is shown as having at least two conductive areas 10a and at least one isolating area 10b. Respectively, electrode 20 consists at least two conductive areas 20a and at least one isolating area 20b. Electrodes 10 and 20 are mounted on the applicator (not shown) in such a way that once the applicator is applied on the skin 50, conductive areas 10a and conductive areas 20a are in contact with the skin. In order to improve the electrical coupling of the electrodes with the skin an impedance matching or conductive material can be applied topically to the skin 50. Electrodes 10 and 20 may be attached to the applicator in such a way that their longitudinal axes are parallel to one other. Moreover, electrodes 10 and 20 are attached to the applicator in a symmetrical way so that each conductive area 10a is paired with a respective conductive area 20a along an imaginary line AA which is perpendicular to the main longitudinal axes of electrodes 10 and 20. This imaginary line establishes the shortest path connecting opposite conductive areas. In other words, electrodes 10 and 20 have an identical physical structure in terms of number and position of conductive areas and isolating areas. However, electrodes 10 and 20 may be wired differently within the applicator so that once connected to an RF generator (not shown) electrode 10 and all of its conductive areas 10a receive one electrical charge while electrode 20 and all of its conductive areas 20a receive an opposite electrical charge.

According to one embodiment of the present invention, electrode 10 may serve as an anode while electrode 20 may serve as a cathode. As further can be seen in FIG. 2, once a RF field is created and delivered into skin 50 by multiple conductive areas 10a and 20a, multiple and separated parallel RF fields 60a are created between working-pairs of the conductive areas lying along the above mentioned imaginary line. Multiple coagulated discrete zones 70a will be created at and around the center of such multiple RF fields 60a so that effectively a deep fractional coagulated-based skin tightening effect is created. Discrete fractional coagulated zones 70a are surrounded by intact healthy tissue sections 80a. Moreover, intact healthy tissue sections 80a may contain blood capillaries like blood capillary 90 which continues to feed adjacent external dermal and epidermal layers to the target treated dermal layer. These intact blood capillaries are essential to keep and maintain the immediate wound healing response post first collagen contraction in order to achieve better skin tightening and collagen remodeling effects.

While electrodes 10 and 20 are shown in FIG. 2 as being substantially parallel to one another, it is understood that the electrodes may be placed in any suitable orientation to one another depending on the desired treatment. Also, while the conductive areas are shown in FIG. 2 as being alternatively placed with non-conductive/isolating areas, it is to be understood that any arrangement of conductive and non-conductive/isolating areas may be used, both within each elongated electrode and with respect of one elongated electrode to another elongated electrode depending on the desired treatment.

In yet another embodiment of the present invention, the applicator may consist of a tissue cooling element which is configured to cool the target tissue deeper to and between electrodes 10 and 20 to allow safe delivery of higher energies into the tissue while keeping the upper layer of the skin intact.

What is claimed is:

1. An apparatus for treating skin tissue with fractional RF energy comprising:
two elongated electrodes;
the elongated electrodes being separated from one another by a distance when placed on the skin tissue;
each of the electrodes having one or more conducting areas and one or more non-conducting areas therealong;
the conducting areas of one elongated electrode being aligned with the conducting areas of a second elongated electrode;
a source of RF energy for providing RF energy to the conducting areas of the elongated electrodes;
wherein, upon application of RF energy to the one or more conductive areas, multiple separated RF fields within the tissue are created to provide fractional coagulation-based skin tightening.

2. The apparatus of claim 1, further comprising a cooling medium applied to one or more of the electrodes for cooling the skin tissue.

3. The apparatus of claim 1 wherein the one or more conducting areas and the one or more non-conducting areas are placed alternatively on the one or more elongated electrodes.

4. The apparatus of claim 1 wherein the elongated electrodes are placed on the skin tissue substantially parallel to one another.

5. A method for treating skin tissue with fractional RF energy comprising:
providing two elongated electrodes;
the elongated electrodes being separated from one another by a distance when placed on the skin tissue;
each of the electrodes having one or more conducting areas and one or more non-conducting areas therealong;
the conducting areas of one elongated electrode being aligned with the conducting areas of a second elongated electrode;
providing a source of RF energy for providing RF energy to the conducting areas of the elongated electrodes;
applying the RF energy source to one or more of the conductive areas;
wherein, upon application of RF energy to the one or more conductive areas, multiple separated RF fields within the tissue are created to provide fractional coagulation-based skin tightening.
